(19) United States
(12) Patent Application Publication
(10) Pub. No.: US 2003/0091524 A1
(43) Pub. Date: May 15, 2003

(54) Hair care composition comprising a conditioning polymer containing polysiloxane-containing radically polymerizable monomer

(76) Inventors: Jian-Zhong Yang, Higashinada-ku (JP); Kiichiro Nakamura, Higashinada-ku (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/228,996

(22) Filed: Aug. 27, 2002

Related U.S. Application Data
(60) Provisional application No. 60/316,635, filed on Aug. 31, 2001.

(51) Int. Cl. A61K 7/06; A61K 7/11

(52) U.S. Cl. 424/70.12

(57) ABSTRACT

Disclosed is a hair care composition that includes a conditioning polymer soluble in fatty alcohols and cationic surfactants that incorporates polysiloxane-containing radically polymerizable monomers, fatty alcohols, cationic surfactants, and an aqueous carrier.

### CROSS REFERENCE TO RELATED APPLICATION

The application claims the benefit of U.S. Provisional application Serial No. 60/316,635 (Case AA570FP), filed on Aug. 31, 2001 in the names of Yang et al.

### TECHNICAL FIELD

The present invention relates to hair care compositions comprising a conditioning polymer comprising a polysiloxane-containing radically polymerizable monomer and a hydrophobic vinyl monomer with an alcohol derived alkyl (meth)acrylate or alkylamine derived alkyl (meth)acrylamide.

### BACKGROUND

Conditioning formulations for hair have a particular thick viscosity that is desirable for such products. These products are based on the combination of a surfactant, which is generally a quaternary ammonium compound, and a fatty alcohol. This combination results in a gel-network structure which provides the composition with the desired rheology. Silicone agents are known to provide conditioning benefits such as smoothness and combing ease to dry hair due to the low surface tension of silicone agents. However, silicone agents are generally water insoluble and decrease the benefits of a surfactant/fatty alcohol gel network for hair conditioning. Often the amount of silicone agents used is limited in amount to minimize interference with the surfactant/fatty alcohol gel network.

Based on the foregoing, there remains a desire to provide hair care compositions suitable for use, which provide improved conditioning benefits to the hair such as smoothness, softness, and reduction of friction, while being easy to apply on the hair.

None of the existing art provides all of the advantages and benefits of the present invention.

### SUMMARY

The present invention is directed to a hair care composition comprising:

A) a conditioning polymer comprising:

(1) a polysiloxane-containing radically polymerizable monomer; and

(2) a hydrophobic vinyl monomer containing a C₁₂-C₄₀ alcohol derived alkyl (meth)acrylate, a C₁₂-C₄₀ alkylamine derived alkyl (meth)acrylamide, or a combination thereof;

B) a fatty alcohol having a C₁₂-C₃₀ alkyl chain and a hydroxyl group,

C) a cationic surfactant having an amine functional group and an alkyl chain; and

D) an aqueous carrier.

The present invention is further directed to a hair care composition comprising:

A) a conditioning polymer comprising:

(1) a polysiloxane-containing radically polymerizable monomer;

(2) a hydrophobic vinyl monomer comprising a C₁₂-C₄₀ alcohol derived alkyl (meth)acrylate, a C₁₂-C₄₀ alkylamine derived alkyl (meth)acrylamide, or a combination thereof; and

(3) an amine functional group-containing (meth)acrylate monomer;

B) a fatty alcohol having a C₁₂-C₃₀ alkyl chain and a hydroxyl group;

C) a cationic surfactant having an amine functional group and an alkyl chain; and

D) an aqueous carrier.

These and other features, aspects, and advantages of the present invention will become evident to those skilled in the art from a reading of the present disclosure.

### DETAILED DESCRIPTION

The present invention is directed to a conditioning composition that contains a conditioning polymer, a cationic surfactant, a fatty alcohol, and an aqueous carrier. The conditioning polymer contains a polysiloxane-containing radically polymerizable monomer, and a hydrophobic vinyl monomer with a alcohol derived alkyl (meth)acrylate or a alkylamine derived alkyl (meth)acrylamine. The hair care composition uses the conditioning polymer with silicone macromer moieties incorporated with a hydrophobic vinyl monomer to increase the solubility of the conditioning polymer in fatty alcohols and cationic surfactants to deliver superior conditioning benefits to the hair.

While the specification concludes with claims, which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”.

All percentages, parts and ratios are based upon the total weight of the hair care compositions of the present invention, unless otherwise specified. All such weights as they pertain to listed ingredients are based on the active level and, therefore, do not include carriers or by-products that may be included in commercially available materials.

The aspects and embodiments of the present invention set forth in this document have many advantages. For example, the hair care compositions of the present invention provide improved conditioning benefits to the hair such as smoothness, softness, and reduction of friction that are easy to apply on the hair.

Conditioning Polymer

The conditioning polymer of the present invention comprises a vinyl monomer with a silicone macromer moiety and a long chain alkyl (meth)acrylate moiety. By incorporating the silicone macromer moiety into a hydrophobic monomer, the negative attributes of a water insoluble silicone agent can be reduced without affecting the gel network formed by the surfactant and the fatty alcohol, both discussed below, in the hair care composition of the present invention. The hair conditioning polymer comprises at least units (A) and (B):

(A) polysiloxane-containing radically polymerizable monomer; and

(B) hydrophobic vinyl monomer containing an alcohol derived alkyl (meth)acrylate, an alkylamine derived alkyl (meth)acrylamide, or a combination thereof.

Polysiloxane-Containing Radically Polymerizable Monomer

The polysiloxane-containing radically polymerizable monomer preferably comprises about 5% to about 40% by weight of the conditioning polymer. The general formula of the polysiloxane-containing radically polymerizable monomer corresponds to the general formula (I)

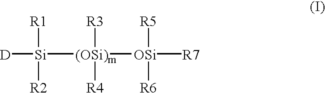

wherein, D represents a radical polymerizing group selected from the group consisting of a vinyl group, a vinylalkyl group, a vinylaryl group, a vinylarylalkyl group, a vinyloxyalkyl group, a (meth)acryloyloxyalkyl group and (meth)acryloylaminoalkyl group; R₁ to R₆, independently represents a hydrogen atom, an alkyl group having from C₁ to C₁₀, or an aryl group; R₇ represents a hydrogen atom, an alkyl group having from C₁ to C₁₀, an aryl group, a vinyl group, a vinylalkyl group, a vinylaryl group, a vinylarylalkyl group, a vinyloxyalkyl group, a (meth)acryloyloxyalkyl group, or a (meth)acryloylaminoalkyl group: and m represents an integer of from about 5 to about 500; more preferably from about 30 to about 300.

Hydrophobic Vinyl Monomer

The hydrophobic vinyl monomer preferably comprises between about 50% and 95% by weight of the conditioning polymer. The hydrophobic vinyl monomer can be either an alcohol derived alkyl (meth)acrylate or an alkylamine derived alkyl (meth)acrylamide.

The alcohol derived alkyl (meth)acrylate preferably contains between about 12 and about 40 carbon atoms, more preferably between about 12 and about 22 carbon atoms, and still more preferably between about 16 and about 18 carbon atoms. Nonlimiting examples cetyl acrylate, cetryl methacrylate, stearyl acrylate, stearyl methacrylate, or any combination thereof.

The alkylamine derived alkyl(meth)acrylamide preferably contains between about 12 and about 22 carbon atoms, more preferably between about 16 and about 18 carbon atoms. Nonlimiting examples include N-cetyl acrylamide, N-cetyl methacrylamide, N-stearyl acrylamide, N-stearyl methacrylamide, or any combination thereof.

Amine Functional Group Containing (Meth)Acrylate Monomer

The conditioning polymer of the present invention may also comprise unit (C) in addition to units (A), (B); (C) being a amine functional group containing (meth)acrylate monomer. The amine functional group containing (meth)acrylate monomer corresponding to the general formula (II):

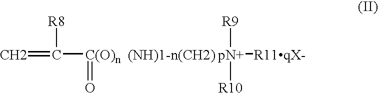

wherein, R₈ represents a hydrogen atom or a methyl group; R₉ and R₁₀, which may be the same or different, each represents an alkyl group having from C₁ to C₂₄, an aryl group, or an arylalkyl group; R₁₁ represents a hydrogen atom, an alkyl group having from C₁ to C₂₄, an aryl group, an arylalkyl group or CH₂—CH(OH)—CH₂—N+R₁₂R₁₃R₁₄ group; R₁₂ to R₁₄, which may be the same or different, each represents a hydrogen, an alkyl group having from C₁ to C₂₄, an aryl group, or an arylalkyl group; X represents anion; n represents 0 or 1, p represents an integer of from 1 to 10; and q represents 1 or 2. In the case that a tertiary amine results, it is preferable that the hair care composition has a pH between 3 and 7.

Preferably, the quaternary amine-containing (meth)acrylate monomer corresponds to the general formula (III):

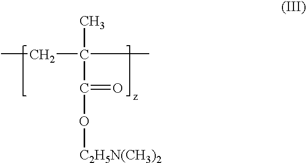

wherein z is such that the quaternary amine-containing (meth)acrylate monomer comprises between about 0% to about 30% by weight of the conditioning polymer. It is further preferable that the use of this preferred general formula (III) be used in a hair care composition with a pH between 3 and 7.

Radically Co-Polymerizable Monomer

The conditioning polymer of the present invention may further include unit (D) in addtion to units (A), (B), and (C); (D) being a radically co-polymerizable monomer comprised of (meth)acrylic acids, (meth)acrylic acid derivatives, (meth)acrylamides, (meth)acrylamide derivatives, or any combination thereof.

Nonlimiting examples of the (meth)acrylic acids and its derivatives include (meth)acrylates, ethyl (meth)acrylate, tetrahydrofurfuryl (meth)acrylate, 2-hydroxyethyl (meth)acrylate, 2-ethoxyethyl (meth)acrylate, and any combination thereof. Nonlimiting examples of the (meth)acrylamide and its derivatives include (meth)acrylonitrile and its derivatives, N,N-dimethyl (meth)acrylamide, N-methyl (meth)acrylamide, N-ethyl (meth)acrylamide, iso-propyl (meth)acrylamide, and any combination thereof.

The radically co-polymerizable monomer may comprise between 0% and 30% by weight of the conditioning polymer.

More preferably, the conditioning polymer corresponds to the general formula (IV):

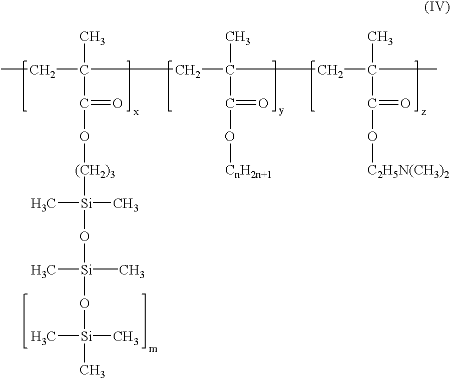

wherein x is such that it comprises between about 5% and about 40% by weight of the conditioning polymer, preferably between about 10% and about 30% by weight of the conditioning polymer; y is such that it comprises between about 50% and about 95% by weight of the conditioning polymer; z is such that is comprises between about 0% and about 30% by weight of the conditioning polymer; m is between about 5 and about 500, preferably between about 30 and about 300; n is between about 12 and about 22, preferably between about 16 and about 18. The weight-average molecular weight of the polymer should be between about 5,000 and about 150,000,000, preferably between about 20,000 and about 700,000, and more preferably between about 50,000 and about 500,000.

The conditioning polymer is preferably included in the hair care composition at a level by weight of from about 0.01% to about 5%, more preferably from about 0.05% to about 2%, and most preferably from about 0.1% to about 1% by weight of the hair care composition.

Methods for making the conditioning polymer disclosed in Japanese patent application number 2001-263597, filed Aug. 31, 2001 by Mitsubishi Chemical Corporation.

Fatty Alcohol

It is believed that fatty alcohols cover the hair surface and reduce friction, thereby resulting in providing smooth feel to the hair and increase the ease of combing. Fatty alcohols are often combined with surfactants, discussed below, to form a gel network to deliver hair-conditioning benefits.

The fatty alcohol is preferably included in the composition at a level by weight of from about 3% to about 14%, more preferably from about 4% to about 10%, and most preferably from about 6% to about 8%.

The fatty alcohols useful herein are those having from about 10 to about 40 carbon atoms, preferably from about 12 to about 22 carbon atoms, more preferably from about 16 to about 22 carbon atoms, and most preferably about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof.

Cationic Surfactant

Among the cationic surfactants useful herein are those corresponding to the general formula (I):

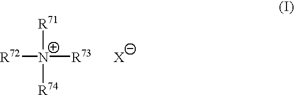

wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbon atoms, or higher, can be saturated or unsaturated. Preferred is when R⁷¹, R⁷², R⁷³ and R⁷⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbon atoms. Nonlimiting examples of such preferred cationic surfactants include: behenyl trimethyl ammonium chloride available, for example, with tradename INCROQUAT TMC-80 from Croda and ECONOL TM22 from Sanyo Kasei; cetyl trimethyl ammonium chloride available, for example, with tradename CA-2350 from Nikko Chemicals, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Amines are suitable as cationic surfactants. Primary, secondary, and tertiary fatty amines are useful. Particularly useful are tertiary amido amines having an alkyl group of from about 12 to about 22 carbon atoms. Exemplary tertiary amido amines include: stearamidopropyldimethylamine, stearamidopropyldiethylamine, stearamidoethyldiethylamine, stearamidoethyidimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyidimethylamine, arachidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidylbehenylamine. Useful amines in the present invention are disclosed in U.S. Pat. No. 4,275,055, Nachtigal, et al.

These amines can also be used in combination with acids such as λ-glutamic acid, lactic acid, hydrochloric acid, malic acid, succinic acid, acetic acid, fumaric acid, tartaric acid, citric acid, maleic acid, and mixtures thereof; more preferably λ-glutamic acid, lactic acid, citric acid. The amines herein are preferably partially neutralized with any of the acids at a molar ratio of the amine to the acid of from about 1:0.3 to about 1:3, more preferably from about 1:0.4 to about 1:2.

More preferable cationic surfactants are tertiary amines or quaternary amines. Most preferable for the cationic surfactant is mono-alkyl amidoamine, mono-alkyl ammonium salt, di-alkyl ammonium salt, PEG(n) alkylamine, or any combination thereof. The cationic alkyl chain length is between about 10 and about 40 carbon atoms, preferably between about 12 and about 22 carbon atoms, more preferably between about 16 and about 18 carbon atoms.

The cationic surfactant comprises between about 0.2% to about 5% by weight of the hair care composition, preferably between about 0.5% and about 4% by weight of the hair care composition, and more preferably between about 1% and about 3% by weight of the hair care composition.

Aqueous Carrier

The hair care compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to about 6 carbon atoms, more preferably ethanol and isopropanol.

Preferably, the aqueous carrier is substantially water; more preferably deionized water is used. Water from natural sources including mineral cations can also be used, depending on the desired characteristic of the product. Generally, the hair care compositions of the present invention comprise from about 20% to about 99%, preferably from about 40% to about 98%, and more preferably from about 50% to about 98% water.

The pH of the present invention is preferably from about 3 to about 7, more preferably from about 3.5 to about 6. Buffers and other pH adjusting agents can be included to achieve the desirable pH.

Additional Components

Additional Silicone Compounds

The hair care compositions of the present invention may contain additional silicone compounds in addition to the conditioning polymer. The additional silicone compounds useful herein include volatile soluble or insoluble, or nonvolatile soluble or insoluble silicone compounds. By soluble means that the additional silicone compound is miscible with the carrier of the composition so as to form part of the same phase. By insoluble what is meant is that the additional silicone compounds forms a separate, discontinuous phase from the carrier, such as in the form of an emulsion or a suspension of droplets of the additional silicone compounds. The additional silicone compounds herein may be made by any suitable method known in the art, including emulsion polymerization. The additional silicone compounds may further be incorporated in the present invention in the form of an emulsion, wherein the emulsion is made by mechanical mixing, or in the stage of synthesis through emulsion polymerization, with or without the aid of a surfactant selected from anionic surfactants, nonionic surfactants, cationic surfactants, and mixtures thereof.

The additional silicone compounds herein are preferably used at levels by weight of the composition of from about 0% to about 10%, more preferably from about 0% to about 5%.

Silicone Fluid

Suitable silicone fluids for use herein include polyalkyl or polyaryl siloxanes with the following structure (I)

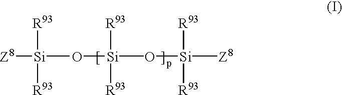

wherein R⁹³ is alkyl or aryl, and p is an integer from about 2 to about 50,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone fluids are polydimethylsiloxane, cyclic dimethyl polysiloxane compounds, polydiethylsiloxane, and polymethylphenylsiloxane. Polydimethylsiloxane, which is also known as dimethicone, and cyclic dimethyl polysiloxane compounds, generally known as cyclomethicone, are especially preferred. The polyalkylsiloxanes that can be used include, for example, polydimethylsiloxanes. These silicone fluids are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

The silicone fluids for use herein will preferably have a viscosity of from about 20 to about 1,000,000 centistokes at 25° C., more preferably from about 10,000 to about 1,000,000, and even more preferably from about 60,000 to about 1,000,000. The viscosity can be measured by means of a glass capillary viscometer as set forth in Dow Corning Corporate Test Method CTM0004, Jul. 20, 1970. Silicone fluids of high molecular weight may be made by emulsion polymerization. Suitable silicone fluids include polyalkyl siloxanes, polyaryl siloxanes, polyalkylaryl siloxanes, polyether siloxane copolymers, and mixtures thereof. Other nonvolatile silicone fluids having hair conditioning properties can also be used.

Particularly suitable silicone fluids herein are non-volatile silicone oils having a molecular weight of from about 200,000 to about 600,000 such as Dimethicone, and Dimethiconol. These silicone fluids can be incorporated in the composition as silicone oils solutions; the silicone oils being volatile or non-volatile or as pre-emulsified oil-in-water emulsions.

Silicone Gum

A nonvolatile dispersed additional silicone compound that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone fluids. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Silicone Resin

Also useful as an additional silicone compound for use herein are silicone resins, which are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present inventions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl™ from Toshiba Silicones.

Modified Silicone Compounds

Other additional silicone compounds that can be used herein include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the modified silicone compounds. These materials are also known as dimethicone copolyols.

Other modified silicone compounds include amino substituted materials. Suitable alkylamino substituted silicone compounds include those represented by the following structure (II)

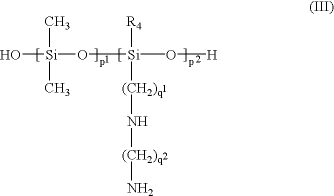

wherein R⁹⁴ is H, CH₃ or OH, p¹, p², q¹ and q² are integers which depend on the molecular weight, the average molecular weight being approximately between 5,000 and 10,000. This polymer is also known as “amodimethicone”.

Nonlimiting examples of commercially available silicone compounds which are useful herein include Dimethicone with tradename DC345 available from Dow Corning Corporation, Dimethicone gum solutions with tradenames SE 30, SE 33, SE 54 and SE 76 available from General Electric, Dimethiconol with tradenames DCQ2-1403 and DCQ2-1401 available from Dow Corning Corporation, Mixture of Dimethicone and Dimethiconol with tradename DC1403 available from Dow Corning Corporation, Dimethicone emulsion with tradename BY22-067 from Dow Corning Chemical Corporation, and emulsion polymerized Dimethiconol available from Toshiba Silicone as described in GB application 2,303,857.

Other Additional Components

The hair care compositions of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition. Such components include, but are not limited to vitamins, amino acids, proteins and protein derivatives, herbal extracts, pigments, dyes, antimicrobial agents, chelating agents, UV absorbers, optical brighteners, perfumes, humectants which are generally water soluble such as polypropylene glycols, additional oily compounds such as mineral oil, additional conditioning agents such as high molecular weight ester oils such as pentaerythritol ester oils with a molecular weight of the compound is from about 800 to about 1200, which are generally water insoluble, and mixtures thereof. The other additional component can be encompassed by visible particles.

A wide variety of other additional components can be formulated into the present inventions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate, antidandruff agents such as zinc pyrithione; and mixtures thereof.

Product Forms

The hair care compositions of the present invention can be in the form of a rinse-off or leave-on products, can be transparent or opaque, and can be formulated in a wide variety of product forms, including but not limited to creams, gels, emulsions, mousses and sprays. Preferably, the hair care compositions of the present invention are rinse-off products.

Method of Use

The hair care composition of the present invention is used in conventional ways to provide the conditioning and other benefits of the present invention. Such method of use depends upon the type of composition employed but generally involves providing hair, which may be dry, wetted, or shampooed, applying an effective amount of the product to the hair, and then drying the hair. Before drying, the hair care composition may be rinsed from the hair (as in the case of hair rinse-off products). “Effective amount” means an amount sufficient enough to provide the conditioning benefits. In general, from about 1 g to about 50 g is applied to the hair, and/or the scalp. During the applying step, the hair care composition may be distributed throughout the hair, typically by rubbing or massaging the hair and scalp, or the composition may be selectively applied to certain parts of the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

All percentages herein are based upon the total weight of the hair care compositions, and all such weight percentages as they pertain to listed ingredients are based on the active level and, therefore, do not include carriers or by-products that may be included in commercially available materials.

| Component | A | B | C | D | E | F |
|---|---|---|---|---|---|---|
| Polymer #1 *1 | 0.3 | 0.6 | 0.2 | — | — | — |
| Polymer #2 *2 | — | — | 0.2 | 0.3 | — | — |
| Polymer #3 *3 | — | — | — | — | 0.6 | — |
| Polymer #4 *4 | — | — | — | — | — | 0.3 |
| PEG-5 cocamine *5 | — | — | 0.5 | 1.0 | — | 13 |
| Citirc acid | — | — | 0.1 | 0.2 | — | — |
| Stearamidopropyldimethylamine *6 | 2.0 | 2.0 | — | — | — | 1.0 |
| L-Glutamic acid | 0.6 | — | — | — | — | 0.3 |
| Lactic acid | — | 1.0 | — | — | — | — |
| Behentrimonium chloride *7 | — | — | 2.0 | 2.0 | 2.0 | — |
| Cetyl alcohol *8 | 2.5 | 2.4 | 5.6 | 1.8 | 2.0 | 3.0 |
| Stearyl alcohol *9 | 4.5 | 3.6 | — | 2.7 | 4.0 | 5.0 |
| Cyclomethicone & Dimethicone *10 | 4.0 | — | 8.0 | 3.0 | 4.0 | 2.0 |
| Dimethicone *11 | — | 1.0 | — | — | — | 2.0 |
| PPG-34 *12 | 2.0 | — | — | 4.0 | — | — |
| Pentaerythritol tetraisostearate *13 | — | 1.0 | — | — | — | 1.0 |
| Mineral oil *14 | — | — | 2.0 | — | — | — |
| Vitamin E | — | — | — | — | 0.05 | — |
| Panthenol *15 | 0.1 | — | — | — | — | — |
| Methyl Paraben | 0.2 | 0.2 | 0.2 | 0.2 | 0.2 | 0.2 |
| Phenoxyethanol | 0.3 | 0.3 | 0.3 | 0.3 | 0.3 | 0.3 |
| EDTA | 0.2 | 0.1 | — | — | 0.1 | 0.2 |
| Disodium EDTA | — | 0.1 | 0.1 | 0.2 | 0.1 | — |
| Benzyl alcohol | 0.4 | 0.4 | 0.5 | 0.4 | 0.2 | 0.4 |
| Perfume | 0.2 | 0.1 | 0.2 | 0.2 | 0.3 | 0.4 |

| | |
|---|---|
| Deionized Water | Add until 100% |

Definitions of Components
*1 Polymer #1: as described in formula (IV) where x = 20 wt. %, y = 80 wt. %, z = 0, m = 150, n = 16, Mw(weight-average molecular weight) = 140,000 available from Mitsubishi Chemical Corporation
*2 Polymer #2: as described in formula (IV) where x = 20 wt. %, y = 80 wt. %, z = 0, m = 150, n = 18, Mw(weight-average molecular weight) = 150,000 available from Mitsubishi Chemical Corporation
*3 Polymer #3: as described in formula (IV) where x = 10 wt. %, y = 80 wt. %, z = 10 wt. %, m = 200, n = 18, Mw(weight-average molecular weight) = 130,000 available from Mitsubishi Chemical Corporation
*4 Polymer #4: as described in formula (IV) where x = 30 wt. %, y = 50 wt. %, z = 20 wt. %, m = 250, n = 16, Mw(weight-average molecular weight) = 200,000 available from Mitsubishi Chemical Corporation
*5 PEG-5 cocamine: Varonic K202 available from Th. Goldschmidt AG
*6 Steraylamidopropyldimethylamine: available from Nikko Chemical
*7 Behentrimonium chloride: available from Sanyo Kasei
*8 Cetyl alcohol: KONOL series available from Shin-nihon Rika
*9 Stearyl alcohol: KONOL series available from Shin-nihon Rika
*10 Cyclomethicone & Dimethicone: 85%/15% mixture of D5 cyclomethicone and dimethicone gum (weight molecular weight of about 400,000 to about 600,000) from General Electric Co.
*11 Dimethicone: EY22-067 dimethicone emulsion from Dow Corning
*12 Polypropyleneglycol (PPG)-34: New Pol PP-2000 available from Sanyo Sasei
*13 Pentaerythritol tetraisostearate: KAK P.T.I. available from Koukyu Alcohol Kogyo Co.
*14 Mineral oil: BENOL available from Th. Goldschmidt AG
*15 Panthenol: available from Roche

### Method of Preparation

The hair care composition of the present invention, is formed by the following process: All percentages are by weight of the final hair care composition. The conditioning polymer is mixed with the fatty alcohol at 65° C. until the mixture becomes transparent. The mixture is added to a cationic surfactant in deionized water at about 85° C. The mixture is maintained at a temperature of about 85° C. for about 10 minutes, until the components are homogenized and no solid particles are observed. The mixture is then gradually cooled to about 55° C. and maintained at this temperature for about 15 minutes. Other ingredients such as cyclomethicone and dimethicone blend, PPG-34, a perfume, a preservative, and other minor components are added after the mixture is cooled to 55° C. The mixture is then further cooled to about 30° C.

Examples A-F, when applied to the hair as a rinse-off product, provide various benefits to the hair such as improved spreadability of the product, hair smoothness, hair softness and reduction of friction.

What is claimed is:

1. A hair care composition comprising:
(1) a conditioning polymer comprising:
a) a polysiloxane-containing radically polymerizable monomer; and
b) a hydrophobic vinyl monomer comprising a C₁₂-C₄₀ alcohol derived alkyl (methl)acrylate, a C₁₂-C₄₀ alkylamine derived alkyl (meth)acrylamide, or a combination thereof;
(2) a fatty alcohol having a C₁₂-C₃₀ alkyl chain and a hydroxyl group;
(3) a cationic surfactant having an amine functional group and an alkyl chain; and
(4) an aqueous carrier.

2. A hair care composition comprising:
(1) a conditioning polymer comprising:
a) a polysiloxane-containing radically polymerizable monomer;
b) a hydrophobic vinyl monomer comprising a C₁₂-C₄₀ alcohol derived alkyl (meth)acrylate, a C₁₂-C₄₀ alkylamine derived alkyl (meth)acrylamide, or a combination thereof; and
c) an amine functional group-containing (meth)acrylate monomer;
(2) a fatty alcohol having a C₁₂-C₃₀ alkyl chain and a hydroxyl group;
(3) a cationic surfactant having an amine functional group and an alkyl chain; and
(4) an aqueous carrier.

3. A hair care composition according to claim 1 or claim 2 wherein the conditioning polymer further comprises other radically co-polymerizable monomer comprising (meth)acrylic acid, (meth)acrylic acid derivatives, (meth)acrylamide, (meth)acrylamide derivatives.

4. A hair care composition according to claim 1 or claim 2 wherein the cationic surfactant amine functional group comprises a tertiary amine, a quaternary amine, or a combination thereof.

5. The hair care composition according to claim 2 wherein the amine functional group containing (meth)acrylate monomer comprises a tertiary amine, a quaternary amine, or a combination thereof.

6. A hair care composition according to claim 4 wherein the cationic surfactant amine functional group comprises a tertiary amine and the hair care composition has a pH less than 7.0.

7. A hair care composition according to claim 5 wherein the amine functional containing (meth)acrylate monomer comprises a tertiary amine and the hair care composition has a pH of less than 7.0.

8. The hair care composition according to claim 1 or claim 2 wherein the conditioning polymer is miscible with fatty alcohols having a C₁₂-C₃₀ alkyl chain and a hydroxyl group and the conditioning polymer has a molecular weight between about 5000 and about 1,500,000.

9. The hair care composition according to claim 1 or claim 2 wherein the conditioning polymer comprises at least 50% by weight of (b).

10. The hair care composition according to claim 1 or claim 2 wherein the conditioning polymer has the following structure (IV): 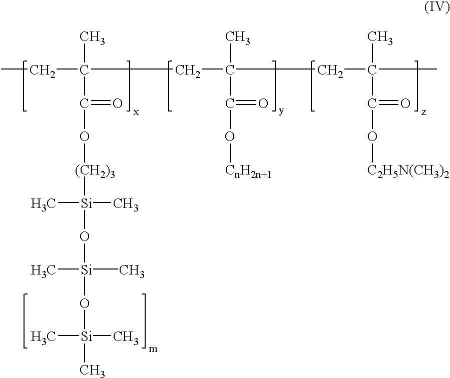
wherein x comprises from about 5% to about 40% by weight of the conditioning polymer; y comprises from about 50% to about 95% by weight of the conditioning polymer; z comprises from about 0% to about 30% by weight of the conditioning polymer; m comprises from about 5 to about 500; n is from about C₁₂ to about C₂₂; and the weight-average molecular weight of the conditioning polymer is from about 50,000 to about 500,000.

11. A hair care composition of claim 9 wherein the fatty alcohol comprises from about 3% to about 14% by weight of the hair care composition, the fatty alcohol further comprising about a C₁₂ to about a C₃₀ alkyl chain and a hydroxyl group; and the cationic surfactant comprises from about 0.2% to about 5% by weight of the hair care composition, the cationic surfactant further comprises steary(amindopropyldimethylamine), behentrimonium chloride, PEG-5 cocamine, or combinations thereof.
